(19) United States
(12) Patent Application Publication
(10) Pub. No.: US 2026/0331244 A1
(43) Pub. Date: Oct. 22, 2026

(54) 3D ORGAN COMPUTED TOMOGRAPHY RADIOLOGY REPORT GENERATION SYSTEM

(71) Applicant: TAIPEI VETERANS GENERAL HOSPITAL, Taipei City (TW)

(72) Inventors: Shih-Jen Chen, Taipei City (TW); Kao-Jung Chang, Taipei City (TW); Shih-Hwa Chiou, Taipei City (TW); Yi-Ping Yang, Taipei City (TW); Cheng-Yi Li, Taipei City (TW)

(73) Assignee: TAIPEI VETERANS GENERAL HOSPITAL, Taipei City (TW)

(21) Appl. No.: 19/555,438

(22) Filed: Mar. 3, 2026

Related U.S. Application Data
(60) Provisional application No. 63/766,039, filed on Mar. 3, 2025.

(51) Int. Cl. G16H 15/00

(52) U.S. Cl. CPC G16H 15/00

(57) ABSTRACT

The disclosure is related to a 3D-organCT dataset and a BrainGPT, a clinically visual instruction-tuned (CVIT) model designed for 3D CT RRG. Since the traditional LLM metrics failed to gauge the diagnostic quality of the RRG, Feature-Oriented Radiology Task Evaluation (FORTE) is proposed to provide an evaluation scheme that captures the clinical essence of the generated reports. The BrainGPT achieves an average FORTE F1-score of 0.71 (degree=0.661; landmark=0.706; feature=0.693, and impression=0.779) and 74% of BrainGPT-generated reports were indistinguishable from human-written ground truth in a Turing-like test. In addition, a comprehensive framework encompassing dataset curation, anatomy-aware model fine-tuning, and the development of robust evaluation metrics for the RRG are established.

### CROSS REFERENCE TO RELATED APPLICATION

This non-provisional application claims the benefit under 35 U.S.C. § 119(e) to U.S. Provisional Application No. 63/766,039, filed on Mar. 3, 2025, all of which are hereby expressly incorporated by reference into the present application.

### FIELD OF THE INVENTION

The invention concerns a 3D organ computed tomography (CT) radiology report generation system.

### BACKGROUND OF THE INVENTION

Artificial intelligence (AI) implementation in modern healthcare has re-invented our day-to-day practice in patient diagnosis^{1, 2, 3}, disease intervention⁴, and clinical research⁵. Although convolution neuron-networks (CNN) had conquered some major tasks in image classification and feature segmentation, the CNN outputs are relatively context-restrictive⁶ and were less apprehensive than a fully written diagnostic report.^{7, 8} In awake to this clinical gap, early report generation models have been established^{9, 10, 11, 12} for chest X-ray (CXR) interpretation.^{13, 14} Whereof, the primary success of LLM-based CXR report generation had fueled interdisciplinary interest to explore human-computer interfaces, wherein multimodal large language models (MLLMs) can be instrumented as an assistant to the medical specialists.¹⁵

MLLMs have demonstrated cutting edge capability in multi-image reasoning and video clip understanding, but these MLLM functionalities were yet fully tested in spatial medical modality. While LLaVA-Med (Microsoft) and Med-PaLM Multimodal (Google Research) have reported their primary success in X-ray and single-slice CT report generation,^{16, 17} essential but open end questions have entailed: (1) how much pathologic features can MLLM describe in a report (2) at what precision (spatial anatomic landmark) level was the lesion attributed in MLLM reports; and (3) whether MLLM could evaluate the degree and size of the lesion. Moreover, current single-slice CT image-text data is manually pre-selected from a 3D data, thus the MLLM is largely tested under a definite-positive lesion included image. This may result in a sharpshooter fallacy in the MLLM testing. Hence, it is essential to create a 3D image set to evaluate MLLM's skills in real-world diagnostics.

3D CT scans of an organ, such as brain, with reports structured in a list-by-list format focusing on differential diagnoses, serve as the first-line diagnostic for diverse intracranial conditions^{18, 19} The brain lesion's degree, size, and location of a brain lesion are paramount for making forming precise diagnoses and guiding subsequent clinical decisions. Therefore, the value of key radiology descriptors outweighs grammatical filler words. In this context, traditional evaluation metrics, which are designed to evaluate short translation^{20, 21}, abstract summary tasks²², and common image captioning²³ cannot capture the clinical essence of brain CT reports.²⁴ In this regard, a pathology keyword retrieval concept was reported in a CheXpert study.^{12, 25} However, the 14-feature retrieval rates cannot reflect the multi-semantic context (inclusive or exclusive; progression or resolution; relative location and size) of the disease nature over an extended clinical course.

Moreover, the uncategorized keyword list in CheXpert may not be transferable from one image modality to different modalities, which profoundly restricts the application value of medical MLLM. Therefore, a robust assessment system was required to improve the clinical essence measurement in MLLM generated radiology reports.

Accordingly, it is desirable to develop a system for MLLM generated radiology reports.

### SUMMARY OF THE INVENTION

Accordingly, the present invention provides a new MLLM radiology report generation system.

In one aspect, the present invention provides a system for generation of 3D organ computed tomography (CT) radiology reports, which comprises a 3D-organ CT dataset and a generative pre-trained transformer, which is adapted for a clinically visual instruction-tuned (CVIT) model designed for 3D-organ CT radiology report generation (RRG), and a processor for feature-oriented radiology task evaluation (FORTE). Since the traditional LLM metrics failed to gauge the diagnostic quality of the RRG, FORTE is proposed to provide an evaluation scheme that captures the clinical essence of the generated reports.

In one embodiment of the invention, the present invention provides a system for generation of 3D-brain CT radiology reports, comprising a 3D-BrainCT dataset (18,885 text-scan pairs) and a generative pre-trained transformer for the dataset, called as “BrainGPT,” which is adapted for a clinically visual instruction-tuned (CVIT) model designed for 3D-brain CT radiology report generation (RRG), and a processor for feature-oriented radiology task evaluation (FORTE).

According to the invention, the BrainGPT achieved an average FORTE F1-score of 0.71 (degree=0.661; landmark=0.706; feature=0.693, and impression=0.779) and 74% of BrainGPT-generated reports were indistinguishable from human-written ground truth in a Turing-like test. In addition, a comprehensive framework encompassing dataset curation, anatomy-aware model fine-tuning, and the development of robust evaluation metrics for the RRG were established.

In one further aspect, the present invention provides a process for implementing a multi-modal large language models (MLLM) in 3D organ CT report generation, comprising
collecting a real-world 3D-organ CT dataset;
training BrainGPT models by applying a visual instruction tuning-based approach;
fine-tuning conditions included regular visual instruction tuning (RVIT):
(i) plain instruction (conveying the model's role as a radiology assistant) and
(ii) in-context example instruction (3-shot examples added to the Plain Instruction), or clinical visual instruction tuning (CVIT):
(iii) template instruction (structured clinical-defined QA templates added to the plain instruction) and
(iv) keyword instruction (categorical guidelines focused on keywords added to the plain instruction).
deriving BrainGPT models (-plain, -example, -template, -keyword) that are fine-tuned to perform CT interpretation with different prior levels of clinical sensibility.

In one embodiment, the organ is brain.

In a yet aspect, the present invention provides a comprehensive framework, which comprises dataset curation, anatomy-aware model fine-tuning, and the development of robust evaluation metrics for the RRG.

To evaluate MLLM readiness in radiology report generation (RRG), related works were surveyed and three objective constraints in hitherto MLLM radiology applications were identified: (1) the most studied CXR modality lacks sufficient lesion diversity to reflect real-world diagnostic challenges, (2) the utmost model capacity was not fully tested in interpreting volumetric scans, and (3) there are no generalized evaluation metrics available to gauge MLLM report information density and fidelity. These unsolved problems combined had crippled the development of an impactful medical MLLM. In the present invention, it was aimed to advance the MLLM adaptability in radiology by addressing the following critical aspects:
(1) to curate a large-scale 3D-brainCT dataset (18,885 text-scan pairs) that consist abundant lesion details including the degree, spatial landmarks, and diagnostic impressions of both neuronal and vascular CT features;
(2) to introduce a clinical visual instruction tuning (CVIT) concept that enhances medical domain knowledge of the open-source Otter foundation model. In this case, our CVIT-augmented BrainGPT model demonstrated multi-image captioning capacity with clinical-sensible interpretation of the volumetric brain CT scans;^{26, 27} in which the diagnostic accuracy and linguistic style of BrainGPT models were externally validated on the CQ500 dataset and 11-physician rater were included to run through a Turing test-like linguistic style evaluation.
(3) to propose a feature-oriented radiology task evaluation (FORTE) evaluation structure to gauge the prospects of the MLLM captions; in which the variables of the FORTE includes four essential keyword components (degree, landmark, feature, and impression) in a diagnostic radiology sentence.

By further inspecting the correlation between the generated content and the evaluation score, it was suggested that preprocessing the MLLM output with sentence pairing and negation removal could enhance the alignment and filter out irrelevant image descriptions.

Overall, the present invention encompasses a holistic content that addresses the objective constraints, implementation details, training methods and future prospects of MLLM application in brain CT and general medical image interpretation.

It is to be understood that both the foregoing general description and the following detailed description are exemplary and explanatory only and are not restrictive of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing description and the following detailed description of the invention will be better understood when reading in conjunction with the accompanying drawings. For the purpose of illustrating the present invention, currently preferred embodiments are shown in the drawings.

FIGS. 1(a)-1(e) provide a schematic overview of instrumenting MLLM to generate radiology reports from 3D brain CT scans. FIG. 1(a) Our customized BrainGPT was trained upon a set of volumetric brain CT image-text pairs, and the training results were evaluated by common language metrics. FIG. 1(b) Before language metrics evaluation, the BrainGPT-generated report was preprocessed by sentence pairing and negation removal. The sentence pairing releases the evaluation constraint of the same-content but different-order false negativity. The negation removal increases feature recall positivity by limiting the range of describing negative findings. FIG. 1(c) A keyword-based Feature-Oriented Radiology Task Evaluation (FORTE) was enrolled to gauge the RRG quality, mainly through referencing a keyword dictionary that described distinct aspects (degree, landmark, feature, and impression) of the lesion. Note that synonyms are accommodated to allow the recognition of a broader array of related terms to address the challenge of lexical variability inherent in clinical reports. FIG. 1(d) The diagnostic accuracy and generalizability of BrainGPT were tested by performing zero-shot captioning on CQ500, an open-source traumatic 3D brain CT external validation dataset. FIG. 1(e) The linguistic style and readiness of the BrainGPT was further evaluated by a Turing test-like assessment in which 11 physicians were asked to distinguish BrainGPT-generated reports from the original ground truths.

FIG. 2 provides the results of employing clinical visual instruction tuning (CVIT) to fine-tune BrainGPT from the baseline Otter model. From the end-to-end Otter foundation structure, we tested four distinct fine-tuning conditions: two of them were regular visual instruction tuning (RVIT)—plain instruction and in example instruction; whereas the other two were clinical visual instruction tuning (CVIT)—template instruction and keyword instruction. To enable multi-image in-context learning, we formatted the data as image-instruction-answer triplets, whereupon the instructions and binarized images were arranged into standardized JSON files using the Otter MIMIC-IT pipeline. Specifically, the Otter framework integrates visual data (instrumenting a frozen CLIP ViT-L/14 encoder) and language data (using the LlaMA-7B large language model) through a trainable perceiver resampler module. In the LlaMA-7B architecture, cross-gated attention layers are added to distribute even focus across volumetric CT scans. The parameters of the remaining modules except for input/output embeddings, are frozen so that the training expenses are minimized. The model efficacy were evaluated via traditional metrics, LLM-as-a-judge evaluation, and a FORTE scoring system tailored for RRG evaluation.

FIGS. 3(a)-3(b) provide the results of assessing visual instruction tuning influence on model performance and characterizing intrinsic CT report structure effects on metric scoring. FIG. 3(a) Traditional language metrics were calculated for each MLLM experiment. While sentence pairing significantly increased the model scoring, statistical significance in two-sided Wilcoxon signed-rank test was also detected between the fine-tuned BrainGPT models and the baseline Otter models; and between the CVIT (Template Instruction and Keyword Instructions) and RVIT (plain instruction and context example instruction) models. FIG. 3(b) MLLM score demonstrated a significant positive shift in the two-sided Pearson correlation coefficient analysis after sentence pairing preprocessing; the pairing-related increase was particularly significant in the measure of CIDEr (TF-IDF-based). (* p<0.05, ** p<0.01, ***p<0.001). CVIT: clinical visual instruction tuning, RVIT: regular visual instruction tuning.

FIGS. 4(a)-4(d) provide the competence of Feature-Oriented Radiology Task Evaluation (FORTE) scoring and the effects of negation removal in RRG. FIG. 4(a) 2D scatter plot documenting the radiology keyword recall discrepancy between the CVIT-conditioned generated reports and the original ground truth. The wording elements were either referred to as the radiology keyword or as the grammatical filler words, among which the CVIT process significantly boosted the keyword usage of BrainGPT models. Note that the differential diagnostic purposed phrase “no” stood out as the top-ranked recall keyword and was further investigated by the negation removal experiments. FIG. 4(b) By further differentiating the keywords into distinct subjects (degree, landmark, feature, impression), we benchmarked inter-model performance and tested the negation removal effects on BrainGPT models. The CVIT and RVIT effects on scoring gains were marked out by red dashed lines, whereas the effect of negation removal scoring gains was marked out by blue dashed lines. Statistical significance was assessed using a two-sided Wilcoxon signed-rank test. FIG. 4(c) The testing of instruction tuning and negation removal was repeated with the assessment of traditional evaluation metrics. Statistical significance for these comparisons was also determined using a two-sided Wilcoxon signed-rank test. FIG. 4(d) Heatmap and 2D scatter plot of the two-sided Pearson analysis showed a distinct evaluation spectrum between the traditional metrics and the FORTE keyword categories. While the traditional evaluation metrics showed high homogeneity, the FORTE evaluation addresses diverse aspects of the radiology description context. (*p<0.05, **p<0.01, ***p<0.001).

FIGS. 5(a)-5(c) provide the results of examining the multi-object multi-slice captioning accuracy of BrainGPT with a trauma-featured CQ500 brain CT external validation dataset. FIG. 5(a) A summarizing bar chart panel was illustrated to display the cranial disease feature composition across our 3D-BrainCT (training and internal testing) dataset and the CQ500 (external validation) dataset. The BrainGPT caption accuracy was tested in three disease features (mass effect, hemorrhage, and midline shift) that came with an ground truth labeling in the original CQ500 dataset. Each of the four BrainGPT models accuracy was displayed by an overhanging line and dot plot. The measured accuracy before and after negation removal were marked by circles and stars, respectively. FIG. 5(b) A head-to-head comparison of distinct BrainGPT-generated reports were showcased in selected brain lesion features (lacunar infarction, subdural hemorrhage; SDH, subarachnoid hemorrhage; SAH), midline shift, and herniation). The precisely mentioned radiology keywords were highlighted in red. An increase trend of keyword usage ascends in correspondence toward the hierarchy of CVIT versus RVIT designs. FIG. 5(c) The multi-slice BrainGPT captioning ability was demonstrated by reporting pathologic conditions spanning across multiple CT scans. In the provided image-text examples, image features are labeled with red arrows, and the corresponding report descriptions are labeled with red context.

FIGS. 6(a)-6(b) provide the Aligning FORTE score utility with physician expert-based evaluation and LLM-as-the-judge. FIG. 6(a) The general and average FORTE score, rather than the traditional metrics, correlate more with the DocLens LLM-as-the-Judge perspective in the two-sided Pearson correlation coefficient analysis. Notably, the correlation increased when sentence pairing pre-processing was applied. FIG. 6(b) Interclass correlation coefficient (ICC) between traditional metrics, FORTE, LLM-based DocLens and the five-item medical expert evaluation was calculated and presented as a heatmap.

FIGS. 7(a)-7(c) provide the results of evaluating the linguistic readiness of BrainGPT reports through a physician participated Turing test. FIG. 7(a) A schematic flowchart of our Turing test survey. Specifically, eleven physicians were asked to distinguish the model-generated report from seven selected report pairs. Each answer accompanies six qualitative reasoning regarding why the decisions were made. A 5-point Likert scale was used to rate the answer confidence, and the participants were asked to address the strength of the rationales (continuity and coherence, familiarity and voice, writing quality and specificity or vagueness). On top of the linguistic compartment of the evaluation, we tested whether the reveal of image context would divert participant impression on the authenticity of the report. FIG. 7(b) The false impression rate of the participant in origin and generated reports; before and after revealing the CT image content. FIG. 7(c) The consideration of linguistic criteria correlates with higher physician success rates.

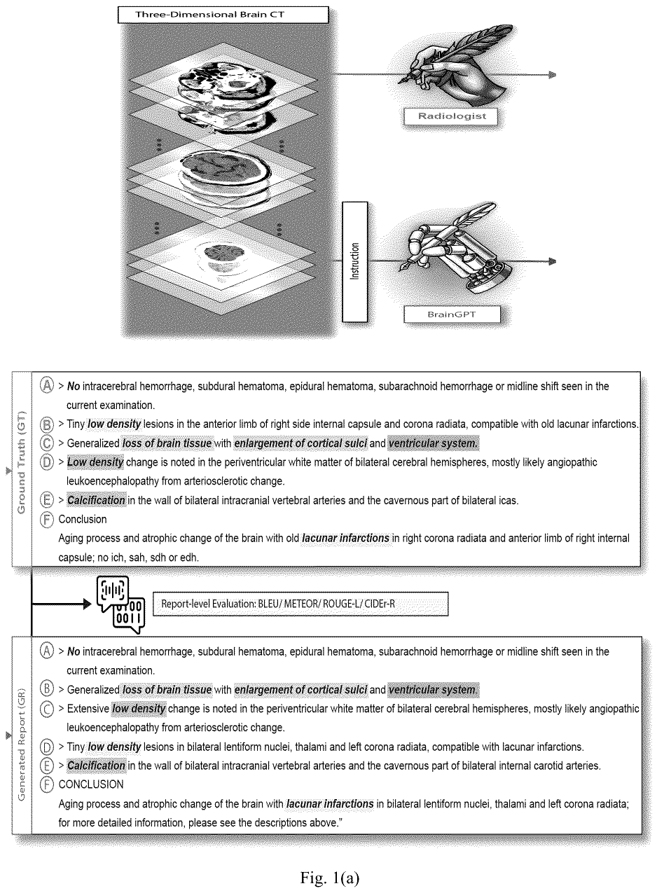

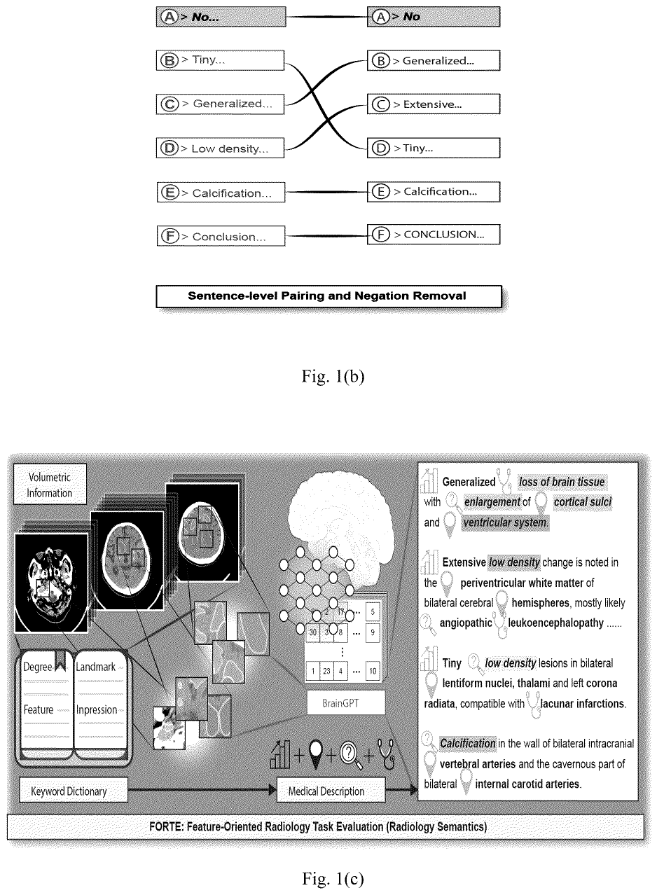

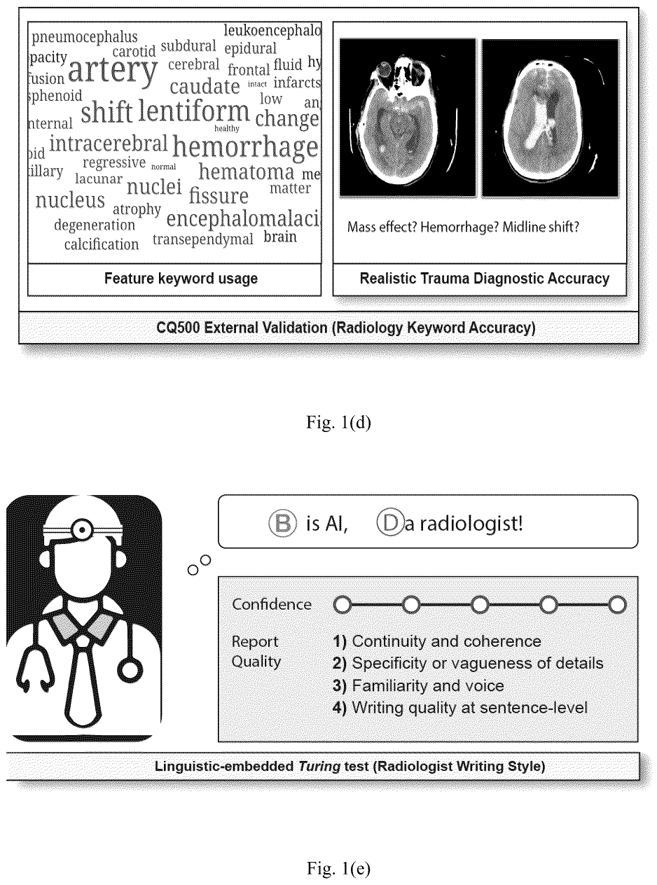

### DETAILED DESCRIPTION OF THE INVENTION

Unless otherwise defined, all technical and scientific terms used herein have the same meaning as commonly understood by those skilled in the art to which the present invention pertains. It should be understood that the terminology used herein is for the purpose of describing specific embodiments only and is not intended to be limiting.

As used herein, the singular forms “a”, “an” and “the” include plural references unless explicitly indicated otherwise. Thus, for example, reference to “a sample” includes a plurality of such samples and their equivalents known to those skilled in the art.

The present invention provides a system for generation of 3D organ computed tomography (CT) radiology reports, which comprises a 3D-organ CT dataset and a generative pre-trained transformer, which is adapted for a clinically visual instruction-tuned (CVIT) model designed for 3D-organ CT radiology report generation (RRG), and a processor for feature-oriented radiology task evaluation (FORTE). Since the traditional LLM metrics failed to gauge the diagnostic quality of the RRG, FORTE is proposed to provide an evaluation scheme that captures the clinical essence of the generated reports.

In one embodiment of the invention, the present invention provides a system for generation of 3D-brain CT radiology reports, comprising a 3D-BrainCT dataset (18,885 text-scan pairs) and a generative pre-trained transformer for the dataset, called as “BrainGPT,” which is adapted for a clinically visual instruction-tuned (CVIT) model designed for 3D-brain CT radiology report generation (RRG), and a processor for feature-oriented radiology task evaluation (FORTE).

According to the invention, the BrainGPT achieved an average FORTE F1-score of 0.71 (degree=0.661; landmark=0.706; feature=0.693, and impression=0.779) and 74% of BrainGPT-generated reports were indistinguishable from human-written ground truth in a Turing-like test. In addition, a comprehensive framework encompassing dataset curation, anatomy-aware model fine-tuning, and the development of robust evaluation metrics for the RRG were established.

According to the present invention, the process for implementing a multi-modal large language models (MLLM) in 3D organ CT report generation, comprises
collecting a real-world 3D-organ CT dataset;
training BrainGPT models by applying a visual instruction tuning-based approach;
fine-tuning conditions included regular visual instruction tuning (RVIT):
(i) plain instruction (conveying the model's role as a radiology assistant) and
(ii) in-context example instruction (3-shot examples added to the Plain Instruction), or clinical visual instruction tuning (CVIT):
(iii) template instruction (structured clinical-defined QA templates added to the plain instruction) and
(iv) keyword instruction (categorical guidelines focused on keywords added to the plain instruction).
deriving BrainGPT models (-plain, -example, -template, -keyword) that are fine-tuned to perform CT interpretation with different prior levels of clinical sensibility.

The present invention also provides a comprehensive framework, which comprises dataset curation, anatomy-aware model fine-tuning, and the development of robust evaluation metrics for the RRG.

### EXAMPLES

### Methods

### Study Design and Oversight

In this study, we trained BrainGPT to generate 3D brain CT reports. Then, we examined the caption efficacy by (1) adapting traditional evaluation metrics (2) proposing a clinical-oriented FORTE metric, (3) applying an external validation CQ500, and (4) conducting the linguistic-embedded Turing test.

### Study Patient

We collected 18,885 brain CT scans (742,501 slices) from 9,689 patients with Alzheimer's Disease (mean age=82.59 years [standard deviation=9.3 years]; 56.4% male) at Taipei Veterans General Hospital in Taipei, Taiwan, between Jan. 1, 2010, and Dec. 31, 2022. All data were collected under institutional review board approval (2023-10-002 BC) of the Taipei Veterans General Hospital. Informed consent was exempted due to the retrospective nature of the data collection and the use of deidentified CT images. The CT images included a variety of common neurology conditions affecting the skull, brain parenchyma, nasal sinuses, and the eye, and were collected by radiologists who routinely obtain CT images and write image reports based on the images and the patient's medical records. Since Alzheimer's Disease is a progressive degenerative condition predominantly seen in the elderly, the dataset includes images of normal brains, past infarcts that still show manifestations, chronic brain conditions, and acute brain lesions.

### Clinical Visual Instruction Tuning (CVIT)

To address the domain constraints of standard MLLM, we conducted multiple end-to-end visual instruction tuning processes on the multi-image mode Otter foundation model, enhancing its applicability to brain CT radiology features.^{26, 27} Based on the Flamingo model, the Otter paradigm connects the LlaMA-7B language encoder and the frozen CLIP ViT-L/14 vision encoder via a trainable perceiver resampler module and multiple cross-attention layers inserted into the LlaMA-7B architecture. Within the original LlaMA-7B structure, all modules except for the input/output embeddings were frozen to reduce training costs. The training duration for each resulting model was 12 hours on two NVIDIA A100 GPUs, achieving 3 epochs.

To facilitate multi-image in-context learning capacity, we formulated the data into image-instruction-answer triplets, with the instruction tokenized and the image enhanced prior to input into the model. We designed four distinct fine-tuning conditions including regular visual instruction tuning (RVIT, Plain Instruction, and In-context example Instruction) and clinical visual instruction tuning (CVIT, Template Instruction, and Keyword Instruction), each corresponding to an adherence hierarchy to clinical essence. We named the final instruction-tuned models BrainGPT-plain, BrainGPT-example, BrainGPT-template, and BrainGPT-keyword.

For each visual instruction tuning process, BrainGPT-plain was fine-tuned using plain instruction, conveying the model's role as a radiology assistant; BrainGPT-example was fine-tuned using in-context example instruction, adopting a 3-shot example approach due to available RAM constraints as based on the work of Singhal et al.;²⁸ BrainGPT-template was fine-tuned using template instruction, involving a structured and predefined set of questions or points that need to be addressed; BrainGPT-keyword was fine-tuned using keyword instruction, focusing on essential areas or categorical guidelines that direct the model's response generation process. The effectiveness of visual instruction tuning was evaluated using a two-sided Wilcoxon signed-rank test. To minimize the influence of reporter writing style, we averaged the reports for each reporter, yielding 27 reporter averages. The Wilcoxon signed-rank test, a non-parametric paired test, was then applied to these related samples to assess whether their population mean ranks differ without assuming a normal distribution. This information has been clearly stated in the revised figure legends.

### Dataset Preparation

Training Dataset. Since the Otter architecture requires image-text pair instances to be of the same size (24 slices), we sampled 365,928 slices from 15,238 scans representing 7,747 patients from a total of 597,335 slices from 15,247 scans representing 7,751 patients for the training process. The system was then tested on 87,312 slices sampled from 145,166 slices of 3,638 CT scans representing 1,938 patients.

External Validation Dataset. The CQ500 dataset, consisting of 1,154 available CT scans from 491 patients, was downloaded from the Qure.ai website.⁵¹ The dataset focuses on image features such as brain parenchyma (plain scans), bone (bone scans), and blood vessels (post-contrast scans). Only non-contrast CT scans with slice numbers between 23 and 40 were selected to build the external validation dataset (n=133). This ensures that slice thickness and details are similar to our training dataset and fit in the Otter framework. Ground truth was based on a read.csv file from the CQ500 dataset, and the majority rule was applied among the three raters to summarize the “Mass effect,” “Hemorrhage event,” and “Midline shift” labels.

### Feature-Oriented Radiology Task Evaluation (FORTE)

We proposed the Feature-Oriented Radiology Task Evaluation (FORTE) framework to capture radiology keywords in terms of Degree (size and intensity), Landmark (location and anatomy), Feature (disease traits and primary findings), and Impression (final diagnosis) of the disease. (For details on the keyword list, see Table 1). This list is not just a compilation of keywords but also includes synonyms, allowing the system to recognize a broader array of related terms that may appear in different contexts, addressing the challenge of lexical variability inherent in clinical reports. The F1 score is calculated for each category, providing a multi-faceted evaluation of the system's performance. Additionally, we compute the Pearson's correlation coefficient (Pearson's r) under two-sided paired 1-test conditions for each FORTE category with traditional metrics, offering a deeper understanding of their applicability and limitations in radiological report evaluation.

TABLE 1
Feature-Oriented Radiology Task Evaluation (FORTE) Keyword Categories and List. This list is not merely a compilation of keywords but also includes
synonyms, enabling the system to recognize a broader array of related terms that may appear in different contexts. For each identified keyword,
the system retrieves its primary representative or returns the keyword itself if no such representative exists. This approach ensures that
semantically similar keywords are treated as equivalent, addressing the challenge of lexical variability inherent in clinical reports.
Degree

| | | | |
|---|---|---|---|
| No | Moderate | Normal/Intact/Healthy | Focal/Territorial/Localized |
| Regressive | Chronic | Inapparent/Nonspecific | Obvious/Apparent |

| | | |
|---|---|---|
| Slight/Subtle/Faint/Minimal/ | Generalized/Extensive/Grossly/ | Abnormal/Atypical/Irregular/ |
| Tiny/Minute/Mild/Low/Thin | Obviously/Clearly/Prominent/Marked | Anomalous |

Landmark

| Posterior | Anterior | Ventral | Lateral | Frontal | Occipital |
|---|---|---|---|---|---|
| Frontoparietal | Occipitoparietal | Parasagittal | Periventricular | Paramedian | Supraclinoid |
| Epidural | Subdural | Subarachnoid | Intracerebral | Subcortical | Supratentorial |
| Ethmoid | Sphenoid | Paranasal | Maxillary | | |
| Gray-white matter | Corpus callosum | White matter/Lacunar | Ventricular/Ventricle | Corona radiata | External capsule |
| Genu | Thalamus/Thalami | Basal ganglia | Striatum | Globus pallidus | Putamen |
| Caudate nucleus/Caudate | | Lentiform nuclei/Lentiform nucleus | | | |
| nuclei/Caudate | | | | | |
| Parenchyma | Insular/Insula | Lobes/Lobe | Centrum semiovale | Venous sinus | Mastoids/Mastoid |
| Vertex Region | Vascular/Artery | Vertebral artery | MCA/Middle cerebral | ICA/Internal carotid arteries/ | |
| | | | artery | Internal carotid siphons | |
| Segment | Scalp | Nasopharynx | Mucus/Mucosal | Lens/Eyes/Eyelid | Foramen magnum |

| | | | |
|---|---|---|---|
| Sulci/Sylvian fissure/Fissure/ | Hemispheres/Hemisphere/Ipsilateral | Bone/Skull | Midline/Falx |
| Cerebral fissure/Sulcus | | | |

Feature

| | | | | | |
|---|---|---|---|---|---|
| Shift | Retention | Congestion | Air-fluid level | Thickening | CSF/Cerebrospinal fluid |
| Density | Crowding | Buckeling | Wedge shape | Lipoma | Hyper dense/Hyperdensity |

| | | | | |
|---|---|---|---|---|
| Metastasis | Infarcts/Ischemic | Hemorrhage/Hematoma | Patches/hypointensity | Dilated/Widening/Enlarged/Enlargement |

| Perifocal edema/Swelling | Hypodensities/Low density/ | Calcified plaque/Calcification/ |
|---|---|---|
| | Attenuation/Hypodensity | Arteriosclerosis/Calcified/ |
| | | Calcification/Calcified-like |
| Cerebrovascular/Angiopathic | Ground-glass opacity/opacity | Atrophy/Atrophic/Degeneration |
| Space occupying lesion/Mass effect | Effusion/Fluid Accumulation | Pneumatized/Air cell/pneumocephalus |

Impression

| | | | | | |
|---|---|---|---|---|---|
| Herniation | Hydrocephalus | Sinusitis | Pneumocephalus | Encephalomalacia | Leptomeningeal seeding |
| Recommend | Craniotomy | Meningitis | Leukoencephalopathy | Cateract | Communicating |
| | | | | | hydrocephalus |
| Meningioma | Thrombosis | Arachnoid cyst | Shunting Catheter | Brain Atrophy | Transependymal effusions |
| Regressive change | Brain Metastasis | Fibrous dysplasia | Wallerian Degeneration | Mastoid effusion | Aging/Aging change/ |
| | | | | | Senile change |

| | | | |
|---|---|---|---|
| SDH/Subdural | EDH/Epidural | IVH/Intraventricular hemorrhage | Aging/Aging change/Senile change |
| hematoma | hematoma | | |

We provided the FORTE keyword JSON files for the Brain CT, chest X-ray, low-dose computed tomography (LDCT), and abdomen CT on our project GitHub page to demonstrate the generalizability of FORTE in facilitating the next generation of medical LLM.

### Traditional Metrics

We compared the clinical evaluation fitness of FORTE against the standard similarity-based evaluation metrics, including BLEU (Bilingual Evaluation Understudy, set range 0-100), METEOR (Metric for Evaluation of Translation with Explicit Ordering, set range 0-100), ROUGE-L (Recall-Oriented Understudy for Gisting Evaluation with Longest Common Subsequence, set range 0-100), and CIDEr-R (Robust Consensus-based Image Description Evaluation, set range 0-1000) (Table 2)^{20, 21, 22, 23} All standard evaluations were executed using the Microsoft Common Objects in Context (MS-COCO) toolkit.⁵²

TABLE 2
Comparison among traditional evaluation metrics and clinically accurate metrics.

| | | | | |
|---|---|---|---|---|
| Evaluation | | | Sentence-level | |
| method | Task/Dataset | Evaluation Method | Comparison | Main Contribution |

Traditional Image Captioning Evaluation Metrics

| | | | | |
|---|---|---|---|---|
| BLEU | Bilingual Evaluation | Quantification of n-gram co- | No | Machine Translation |
| | Understudy | occurrence frequencies | | |
| | | (unigram = single word) | | |
| METEOR | Metric for Evaluation of | Word-to-word matching | No | Machine Translation |
| | Translation with | with precision, recall, and | | |
| | Explicit Ordering | penalty calculations | | |
| ROUGE-L | Recall-Oriented | Longest Common | No | Machine Translation/Document |
| | Understudy for | Subsequence (LCS) | | Summarization |
| | Gisting Evaluation | | | |
| | with Longest | | | |
| | Common Subsequence | | | |
| CIDEr-R | Robust Consensus-based | Cosine similarity of the | No | General Image Captioning |
| | Image Description | frequency-inverse | | |
| | Evaluation | document frequency | | |
| | | (TF-IDF) vectors | | |

Clinically Accurate Metrics

| | | | | |
|---|---|---|---|---|
| CheXpert | Chest X-ray Feature | Radiology Term F1-score | No | An automatic labeler to extract radiology terms |
| | Labelling | | | from free-text radiology reports |
| Doclens | Radiology report | Chain-of-thought (CoT) | Yes | Evaluation across three medical text tasks: |
| | summarization | Prompting GPT Evaluation | | clinical note generation, radiology report |
| | (MIMIC-III) | (Sentence-level | | summarization, and patient question |
| | | Precision and Recall) | | summarization. |
| FineRadScore | Chest X-ray report generation | Few-shot Prompting GPT | Yes | Sentence-level corrections with severity ratings |
| | (MIMIC-CXR dataset) | Evaluation (Error counts) | | and qualitative feedback. |
| RadFact | Chest X-ray report | Few-shot Prompting GPT | Yes | Grounded sentence-level evaluation with spatial |
| | generation (Multiple | Evaluation (Sentence-level | | correspondence. |
| | dataset) | Precision and Recall) | | |
| FORTE | Brain CT report | Radiology Term F1-score | Yes | Comprehensive framework for assessing |
| | generation (3Dbrain-CT) | | | clinical relevance of captions using four key |
| | | | | categories: degree, landmark, feature, and |
| | | | | impression. |
| CheXprompt | Chest X-ray report | Few-shot Prompting GPT | No | An evaluator capable of classifying six |
| | generation (MIMIC- | Evaluation (Error counts) | | categories of errors: false positive findings, |
| | CXR dataset) | | | omissions, location errors, severity errors, and |
| | | | | comparison inconsistencies. |

Additionally, to address the list-by-list structure, diverse paraphrasing, and differential diagnosis-oriented negation description of brain CT reports, we incorporated “sentence pairing” and “negation removal” before applying the evaluation formulas. The “sentence pairing” process was inspired by BERTScore, which leverages contextual embeddings and cosine similarity to achieve better semantic matching than traditional n-gram-based methods.⁵³ Previous literature showed the BERT embeddings⁵⁴ capture rich semantic information and are widely recognized as effective for sentence similarity tasks.^{55, 56, 57} Cosine similarity, which measures the token-level contextual embeddings of reference and candidate sentences, is widely recognized as an effective generation metric and applied in the CheXbert vector similarity metric.⁵⁸ Specifically, the reports were embedded and vectorized using the all-mpnet-base-v2 model from the Sentence Transformer library before pairing and scoring. On the other hand, the “negation removal” process filtered any ground truth/generated report pair with the word “no” with the “search” module under “re” package. By these means, sentence pairing releases the sequential constraints of disease descriptors, and negation removal reduces the false positives in evaluation reports.

### LLM-as-a-Judge for Medical Report Validity

To assess the performance of BrainGPT and our proposed metric, FORTE, we conducted an evaluation using both an LLM-as-a-judge framework and a formal clinical accuracy evaluation by physicians. For the LLM-as-a-judge evaluation, we utilized the DocLens metric, which employs GPT-4o mini to extract claims from generated reports, verify their presence in ground-truth reports, and compute a precision score for the generated content. (for the prompt used, please refer to Table 3 for the claim and precision examples)²⁹.

TABLE 3
Prompts Used for DocLens Evaluation.

Prompt for Claim Generation
The following is a report. Please divide it into several claims and ensure there is no overlap or omission between
claims. Provide the claims as a list in the following format:
[\“claim1\”, \“claim2\”, \“claim3\”, . . . ].
Report:
Prompt for Claim Precision Precision Computation
Please act as a fair judge and evaluate whether the claims are fully contained within the provided imaging report.
When comparing the claims to the imaging report, you may account for synonyms and paraphrasing. For example,
words or phrases with the same meaning (e.g., “mild” and “minimal”) or rephrased statements can be considered
valid matches. For each claim, output “1” or “0” where “1” means the claim is fully contained and “0” means it
is not. Also provide an explanation for your decision. Output the result as a list of dictionaries in text format (not
JSON). The format should be: [{“claim”: “the original claim1”, “explanation”: “the reason why the entailment
prediction is made”, “entailment prediction”: 1 or 0}, {“claim”: “the original claim2”, “explanation”: “the reason
why the entailment prediction is made”, “entailment prediction”: 1 or 0}, . . . ]
Claims:

Five models were evaluated, including BrainGPT-plain, BrainGPT-example, BrainGPT-template, BrainGPT-keyword, and the baseline Otter model. Additionally, we compute the Pearson's correlation coefficient (Pearson's r) under two-sided paired t-test conditions for DocLens score with the FORTE categories and traditional metrics, offering a deeper understanding of their applicability and limitations in radiological report evaluation. Pearson correlation analyses were also performed between DocLens precision and the FORTE categories, with and without sentence pairing preprocessing, to examine the impact of this step on the correlation.

For the clinical evaluation, a physician survey was constructed using 50 samples randomly selected from the lowest and highest one-third of sentence pairs (25 samples each) based on FORTE average scores. The survey employed the RADPEER framework, developed by the American College of Radiology (ACR), and included five evaluation criteria: “Diagnostic Accuracy,” “Linguistic Quality,” “Radiologic Detail Information,” “Density Proportion,” and “Soundness Equivalence.”^{33, 59} To maintain focus on these aspects, only the AI-generated reports and their corresponding ground truth reports were provided to the physicians. Intraclass correlation coefficients (ICCs) were calculated to measure the reliability of human evaluations across these criteria. Additionally, RADPEER scores were dichotomized using arithmetic means as thresholds to compute Cohen's kappa coefficients with DocLens scores.

### Linguistic-Embedded Turing Test

To examine whether the BrainGPT CT reports recapitulate the linguistic texture of radiologist reports, we conducted a Turing test involving physicians and radiologists. Each participant was asked to distinguish BrainGPT reports from radiologist reports. The study was structured around four measuring axes:
(1) Turing test: Can physicians tell the difference between BrainGPT reports and radiologist reports?
(2) Confidence rate: How confident are the reviewers in their ratings?
(3) Inter-leaved dependency: Do physicians alter their assessments and confidence rates after reviewing the original CT scans?
(4) Linguistic criteria: What is the linguistic rationale behind physicians' impressions?³⁴

To explore the aforementioned questions, we collected survey and semi-structured rationale interview data. The physician survey was composed of six caption pairs, each comprising a BrainGPT report and a radiologist report. These examples included diverse disease instances including lacunar infarct, subdural hemorrhage, brain atrophy, and midline (cerebral falx) shift, thereby encompassing a range of both acute and chronic cerebral alterations for expert evaluation.

### Data Availability

The TPEVGH data used in this study cannot be released due to IRB regulations. The CQ500 dataset used for external validation is available in the Qure.ai database and can be accessed via the provided reference.⁵¹ Note that any further distribution of datasets is subject to the terms of use and data-sharing agreements stipulated by the original creators.

### Code Availability

All code used for experiments in this study can be found in a GitHub repository (https://github.com/charlierabea/FORTE). The repository also contains a Huggingface model weight link to our best model (https://hugaingface.co/Charliebear/BrainGPT) and the FORTE keyword JSON files (Chest X-ray/Brain CT/Low-dose CT/Abdomen CT).

### Results

Training BrainGPT with Clinical Visual Instruction Tuning

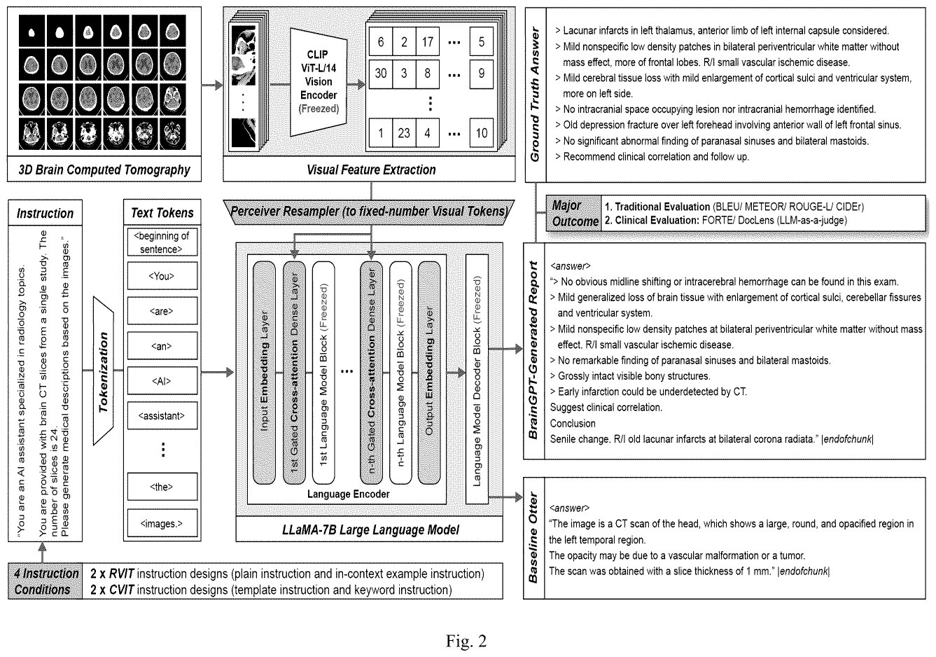

To implement medical MLLM in 3D brain CT report generation, we collected a real-world 3D-BrainCT dataset and trained BrainGPT models by applying a visual instruction tuning-based approach (FIG. 2). Our fine-tuning conditions included regular visual instruction tuning (RVIT): (1) plain instruction (conveying the model's role as a radiology assistant) and (2) In-context example instructions (3-shot examples added to the Plain Instruction), or clinical visual instruction tuning (CVIT): (3) template instruction (structured clinical-defined QA templates added to the plain instruction) and (4) keyword instruction (categorical guidelines focused on keywords added to the plain instruction). As a result, we derived four BrainGPT models (-plain, -example, -template, -keyword) that are fine-tuned to perform CT interpretation with different prior levels of clinical sensibility.²⁵

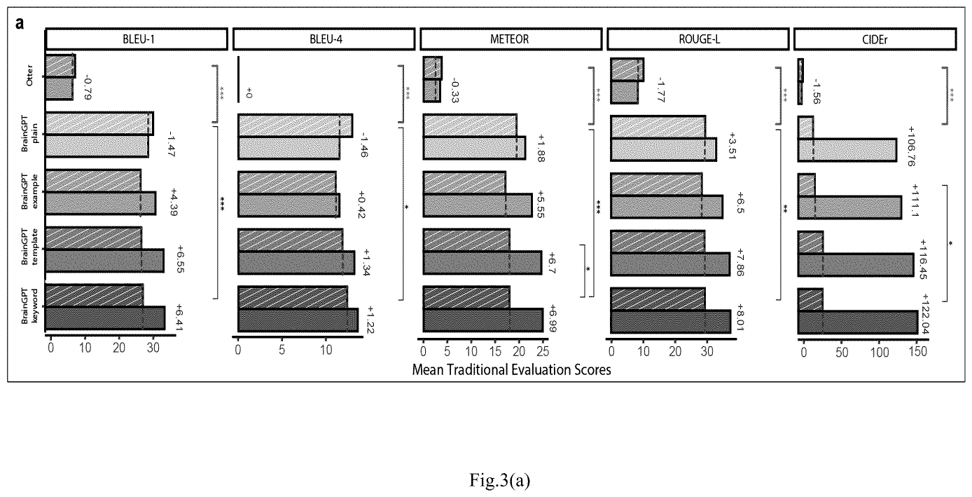

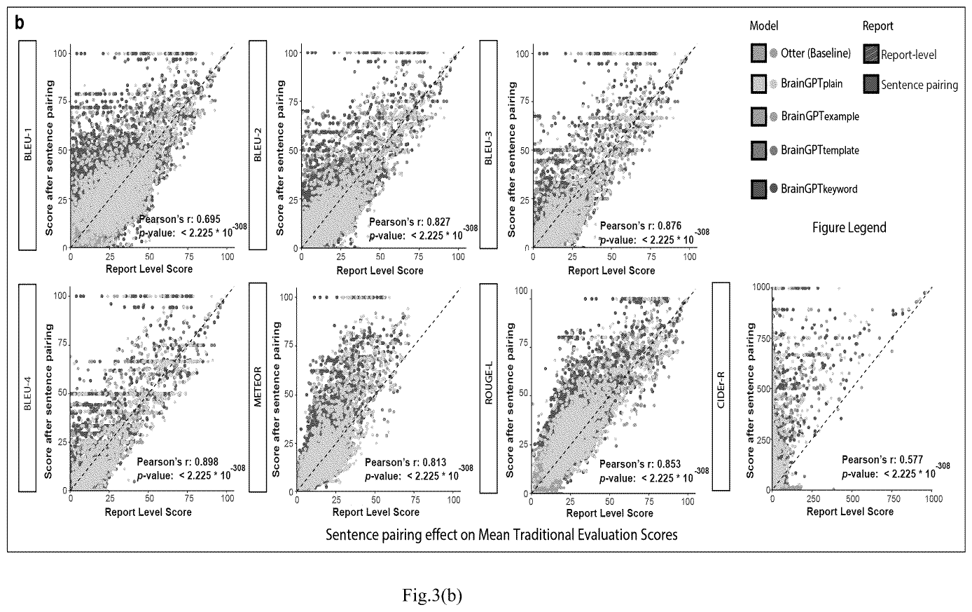

At the whole report level, all of the four fine-tuned BrainGPT models outperformed the baseline Otter model in traditional metrics (Two-sided Wilcoxon signed-rank test p<0.01, FIG. 3a, for complete statistics). In particular, the baseline Otter model scored the lowest in the BLEU-4 (0) and CIDEr-R (5.9) metrics, indicating that the baseline model-generated CT report had low n-gram overlap and underfit semantic term usage frequency. In addition, we observed a mismatch between the conventional metric and the report quality by reviewing generated reports. However, no traditional metrics, except CIDEr-R, rated the BrainGPT performance in accordance with the crescendo elaborateness of RVIT and CVIT (Two-sided Wilcoxon signed-rank test p>0.05 across most visual instruction tuning conditions. Therefore, we demonstrated that traditional metrics are inherently insensitive to the clinical essence of the generated radiology reports.

### Sentence Pairing

To investigate whether the list-by-list report architecture of brain CT images accounts for low traditional metric scores, we apply sentence pairing to decompose the multisentence paragraph into smaller semantic granularity. As a result, the process of sentence pairing increased traditional metric scores by an average of 5.28 points in METEOR, 6.48 points in ROUGE-L, and an astonishing 114 points in CIDEr-R (FIG. 3a). The traditional metric scores for every BrainGPT model were significantly improved across most traditional metrics (p<0.001, as shown in FIG. 3b). In addition, the score distribution indicated a significant linear correlation (two-sided Pearson correlation coefficient r>0.7) in BLEU-2, BLEU-3, BLEU-4, METEOR, and ROUGE-L with statistical significance (p<0.001). However, apart from the other n-gram-based traditional metric, the CIDEr-R score gained a more positive shift from the process of sentence pairing (r=0.577).

Moreover, we observed a positive correlation between advanced CVIT and increased traditional metric scores. In particular, the CIDEr-R score depicted the most prominent ascending trend across the hierarchically fine-tuned BrainGPT-plain (125.86), BrainGPT-example (132.38), BrainGPT-template (147.92), and BrainGPT-keyword (153.3). This sentence pairing procedure not only relieves the sequential constraint between the report input and generated output but also accentuates the clinical instruction design value in the generation of the line-by-line differential diagnosis reports.

### Feature-Oriented Radiology Task Evaluation (FORTE)

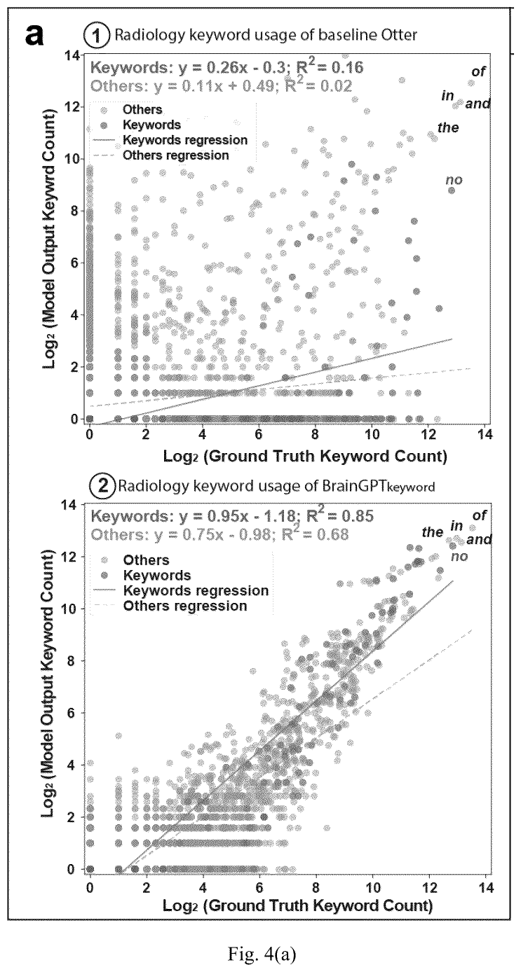

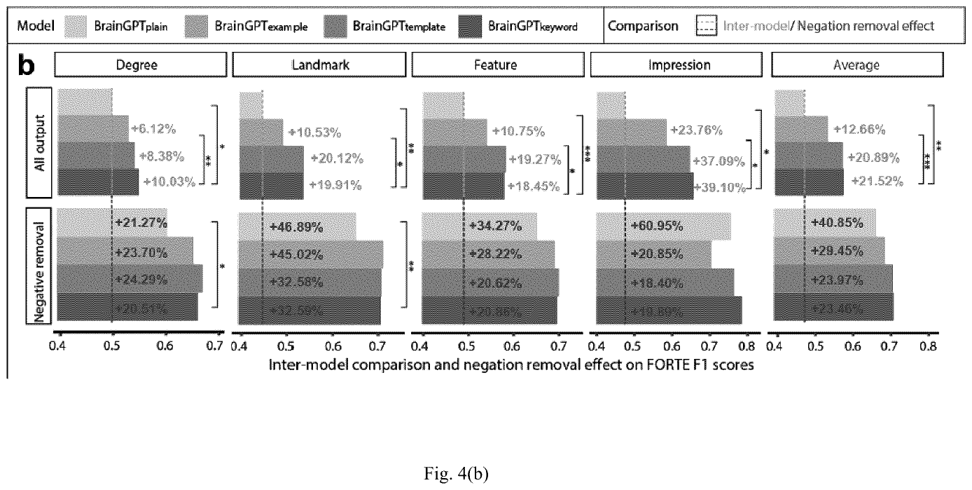

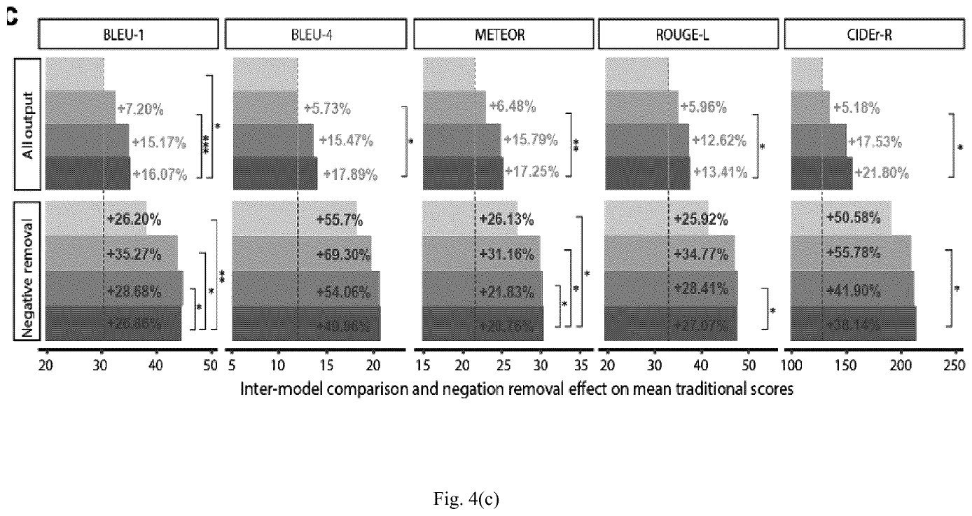

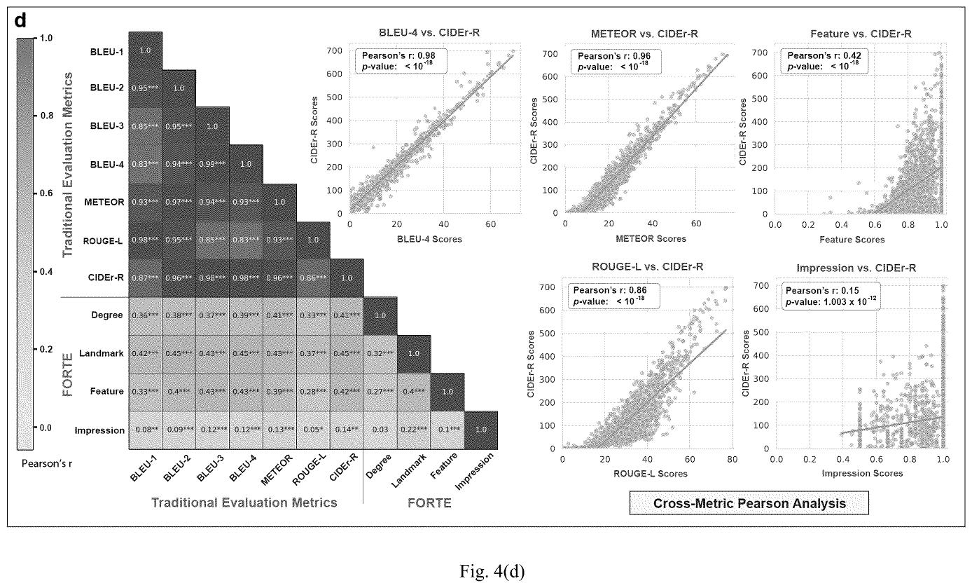

Given that CIDEr-R captures the hierarchical clinical essence across visual instruction tuning conditions, we hypothesized that its frequency-inverse document frequency (TF-IDF) component reacts to the usage of rare radiology keywords in reports. Therefore, we analyzed the term frequency in both the ground truth and test outputs (FIG. 4a). We found that the baseline Otter model had a low recall rate for keywords, whereas the BrainGPT models, after visual instruction tuning, demonstrated significant radiology keyword usage in image captioning. Notably, the BrainGPT-keyword (FIG. 4a: y=0.95x−1.18; r²=0.85) maintained a high recall rate across varying frequencies of radiology keywords.

On the basis of the keyword frequency experiment and a thorough analysis of the CheXpert classifier, we consider that a clinically-sensible evaluation metric should encompass a structured keyword extraction concept that (1) addresses the multi-semantic context of radiology reports, (2) allows the recognition of synonyms to detect a broader array of related terms and (3) can be transferred across multiple modalities. In this regard, we introduced the feature-oriented radiology task evaluation (FORTE). This method gauges the medical content of the generated report by focusing on the density of radiology information. We categorized radiology keywords and their synonyms into degree, landmark, feature, and impression subsets to perform a multi-faceted evaluation of the system's performance.

By evaluating the F1 score of each FORTE-measured keyword category (see FIG. 4b), we noted that the advanced CVIT models, BrainGPT-template and BrainGPT-keyword, scored higher average F1 scores than the RVIT models did. (Wilcoxon signed-rank test p-value <0.05 in most categories.) Specifically, the BrainGPT-keyword captured the most radiology details, with FORTE F1 scores of 0.548, 0.533, 0.574, and 0.649. These collective results indicate that clinical instruction-tuned BrainGPT models generate brain CT reports with a higher level of radiology term usage and better alignment with original diagnostic reports.

Comparison of FORTE with Traditional Evaluation Metrics

To determine whether FORTE reveals the qualities of reports revealed by traditional metrics, we conducted a two-sided Pearson correlation coefficient analysis to compare FORTE with traditional metrics (FIG. 4d). We observed that traditional metrics had high intracorrelations (r>0.7, p<0.001), but a lower correlation with FORTE (r<0.5, p<0.001) than with the FORTE. In addition, the four FORTE domains (degree, landmark, feature, and impression) had lower intracorrelations (r<0.5, p<0.001 compared with traditional metrics). This comparison indicated that the FORTE addresses broader and distinct disease aspects than those traditional metrics depict.

### Negation Removal: Effect of “Interpretation Spree”

Upon further review of keyword usage frequency, we found that “no” was notably among the most frequently used words, along with “of,” “and,” “in,” and “the.” This contradicts the common belief of “reporting bias,” where it is assumed that language models, similar to laypersons, are less likely to mention negative features. Clinically, radiologists often focus on context-oriented descriptions to “rule out” diagnostic targets, a practice not mirrored by BrainGPT. Consequently, BrainGPT's reports were overlaid with excessive negative descriptions rather than the concise, context-specific language used by radiologists, a phenomenon we termed the “Interpretation spree.”

To address this, we applied negation removal to limit descriptions to positive findings. (FIG. 4c) The average score increases were as follows: BLEU-1=29.25%, BLEU-2=34.9%, BLEU3=50.54%, BLEU-4=57.26%, METEOR=24.97%, ROUGE L=29.04%, CIDEr-R=46.6%, with statistical significance in a two-sided Pearson correlation coefficient analysis.

Additionally, negation removal improved the BrainGPT FORTE keyword average F1 score by 0.118 for degrees, 0.194 for landmarks, 0.139 for features, and 0.163 for impressions (see FIG. 4b). Negation removal led to an overall average F1 score gain of 0.153, with the best instruction-tuned model, BrainGPT-keyword, seeing its F1 score rise from 0.576 to 0.71. To explore the effects of negation removal on keyword distribution more deeply, we inspected the percentage of sentences containing four elements before and after “Negation Removal.” The initial FORTE evaluation showed all keyword categories were present in 20.9/6-27.4% of the BrainGPT-generated reports, whereas the number decreased to 3.3%-6.2% after negation removal. On the other hand, “degree and landmark” was present in 29.3/6-35.3% of the BrainGPT-generated reports, whereas the number increased to 41.6%-55% after negation removal. This may be due primarily to the fact that negation removal resulted in the depletion of mostly impression keywords. The effect corresponds with the conciseness of radiology reports that the impression is succinct, and every sentence conveys a specific keyword combination including but not limited to all four elements for the purpose of differential diagnosis.

Together, we demonstrated that negation removal allowed evaluation metrics to focus on positive findings, enhanced average traditional scores and FORTE F1 scores, and avoided sparse and off-topic impressions in reports.

### Assess BrainGPT Generalization Via CQ500 External Validation

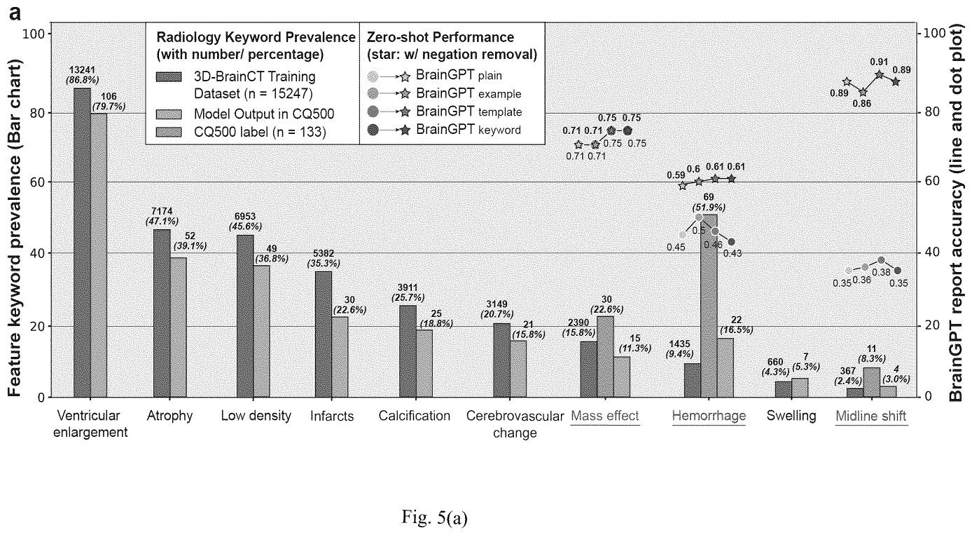

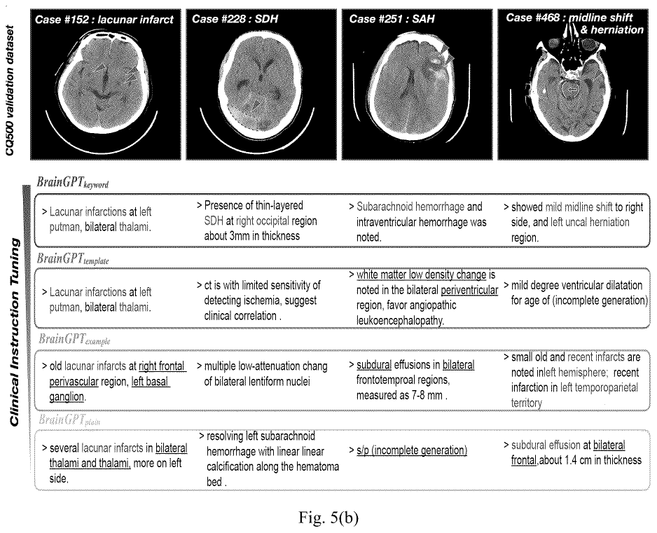

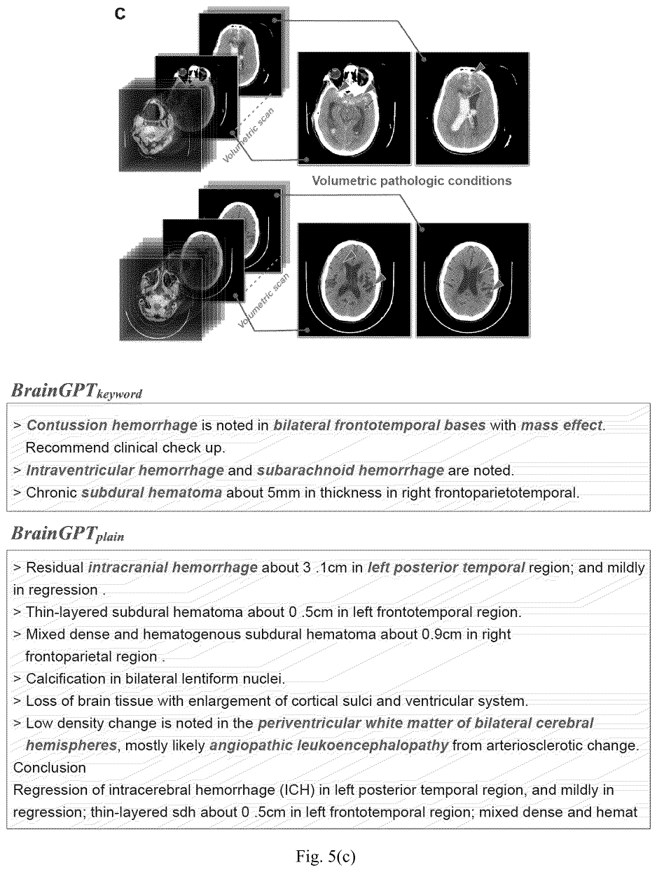

We conducted zero-shot external validation on the CQ500 brain CT dataset (n=133) to assess BrainGPTs ability to perform report captioning under generalized clinical conditions. We estimated the clinical keyword retrieval rate by comparing the BrainGPT reports to the ground truth labels of the CQ500 CT scans (FIG. 5a). Notably, BrainGPT mentioned keywords (e.g., ventricular enlargement, atrophy, infarction, and mass effect) in the generated reports for the CQ500 validation dataset with frequencies similar those of the training dataset (3D-BrainCT). These zero-shot captioning results indicated that BrainGPT included the CT report phraseology and writing structure for the differential diagnosis of brain disease.

CQ500 is an intracranial hemorrhage (ICH) dataset encompassing “mass effect,” “hemorrhage,” and “midline shift” features, which were less indexed in our geriatric population 3D-BrainCT dataset. Nevertheless, the four instruction-tuned BrainGPT models achieved fair accuracy in reporting mass effects (0.71-0.75), midline shifts (0.35-0.38), and hemorrhagic events (0.43-0.5). Furthermore, consistent with our previous findings, negation removal (aligning with differential diagnosis interest) significantly increased the reporting accuracy for midline shift (0.86-0.91) and hemorrhagic events (0.59-0.61). Notably, in terms of diagnosing “mass effect,” “hemorrhage,” and “midline shift,” the advanced CVIT models (BrainGPT-template and BrainGPT-keyword) generally outperformed the RVIT fine-tuned models (BrainGPT-plain and BrainGPT-example). This suggests that CVIT instructions act as a premodel rectifier, enhancing the focus on valuable clinical etiologies.

To further investigate diagnostic credibility, we examined report results for lacunar infarct, subdural hemorrhage (SDH), subarachnoid hemorrhage (SAH), and midline shift with mass effects (FIG. 5b). Interestingly, in the lacunar infarction case, the BrainGPT-example accurately identified the hypodense old lacunar infarct in the putamen and thalamus regions. However, “putamen” was misspelled as “putmen,” likely due to a typo learned from the original training report. The model also generated successful captions for brain lesions, including SDH at the right occipital region, SAH, and left uncal herniation. To examine whether BrainGPT was subject to the sharpshooter fallacy in 3D CT scans, we isolated multiobject cases with different lesion features in various CT images (FIG. 5c). We noted that in some multi-lesional brain CTs, the BrainGPT reports could recognize distinct hemorrhage subtypes (contusion, intraventricular and subarachnoid hemorrhage) with a sparse comprehension of the lesion location (frontotemporal lobe). In another case, BrainGPT described a regressing chronic ICH and a periventricular angiopathic leukoencephalopathy with white matter hypodensity. These multiobject, multislice caption reports demonstrated BrainGPTs potential to provide differential diagnoses for hemorrhages and hypodense image features.

### LLM-as-a-Judge for Medical Report Validity

Our BrainGPT scored robust performance in both traditional evaluation metrics and FORTE. To further validate the clinical reliability of BrainGPT and FORTE metrics, we leveraged the LLM-based DocLens as an extrapolating referee to measure the fine-grained medical details in the BrainGPT generated reports and the efficacy of FORTE.²⁹ Interestingly, by comparing the DocLens claim precision scores and the FORTE scores in the status pre- (0.102 vs 0.102) and post-visual instruction tuning (0.589 vs 0.539), we noted that the FORTE scoring framework aligns with the LLM—as the judge evaluations.

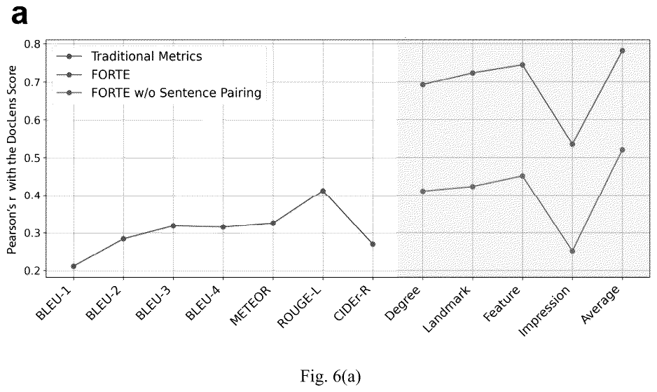

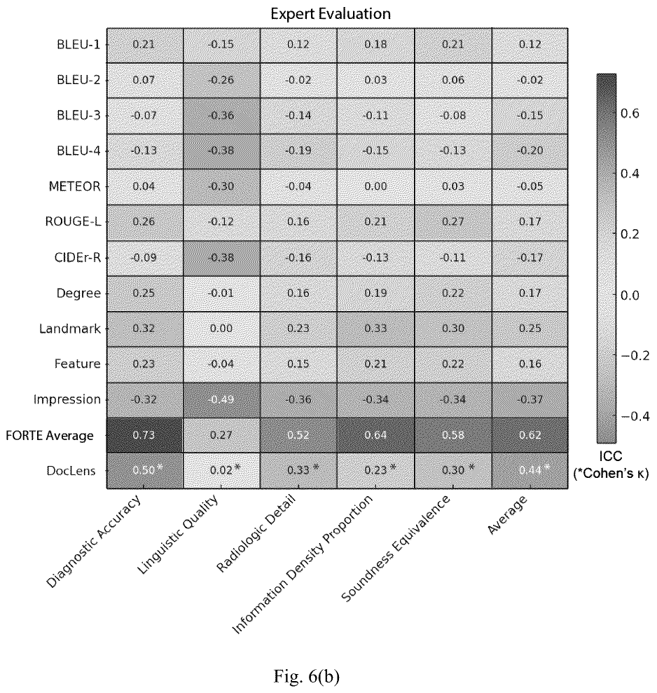

To examine the semantic coherence between the FORTE and LLM-based evaluations, we conducted two-sided Pearson correlation analysis between DocLens score and all aforementioned metrics. Notably, we observed that Doclens is highly correlated with FORTE (r=0.78, p-value<0.001), but is poorly correlated with traditional metrics (r<0.4, p-value <0.001). The improved coherence between DocLens scores and FORTE metrics following the application of sentence pairing highlights the importance of this evaluation preprocessing step (FIG. 6a) In summary, our results demonstrated that traditional metrics fall short to address the semantic complexity in medical reports, and the FORTE scoring concept work as efficient as the state-of-the-art DocLens evaluation but in a computation-light manner.

To accredit the whole BrainGPT-generation and FORTE-evaluation framework with human expert approval, we constructed a 10-point likert scale rating by 2 physicians reading 50 random sampled captions. The surveyed dimensions were set by recombination the essence of radiological RADPEER, linguistic readability and factual considerations.^{30, 31, 32, 33} The evaluation compared AI-generated reports with their corresponding ground truth reports across five key criteria: “Diagnostic Accuracy,” “Linguistic Quality,” “Radiologic Detail Information,” “Density Proportion,” and “Soundness Equivalence.” We then computed the intraclass correlation coefficients (ICC) between the five criteria (FIG. 6b) and the FORTE average F1 score, which derived a moderate correlation between human expert opinion and proposed FORTE metrics: 0.73 (Diagnostic Accuracy), 0.27 (Linguistic Quality), 0.52 (Radiologic Detail Information), 0.64 (Density Proportion), 0.58 (Soundness Equivalence), and 0.62 overall. In parallel, we also noted a moderate correlation between DocLens and human expert opinion in terms of “Diagnostic Accuracy”, and a mild correlation in the remaining rated items. In conclusion, BrainGPT generated accurate and informative radiologic reports, whilst the proposed FORTE effectively denotes the medical essence within the report.

### Linguistic-Embedded Turing Test

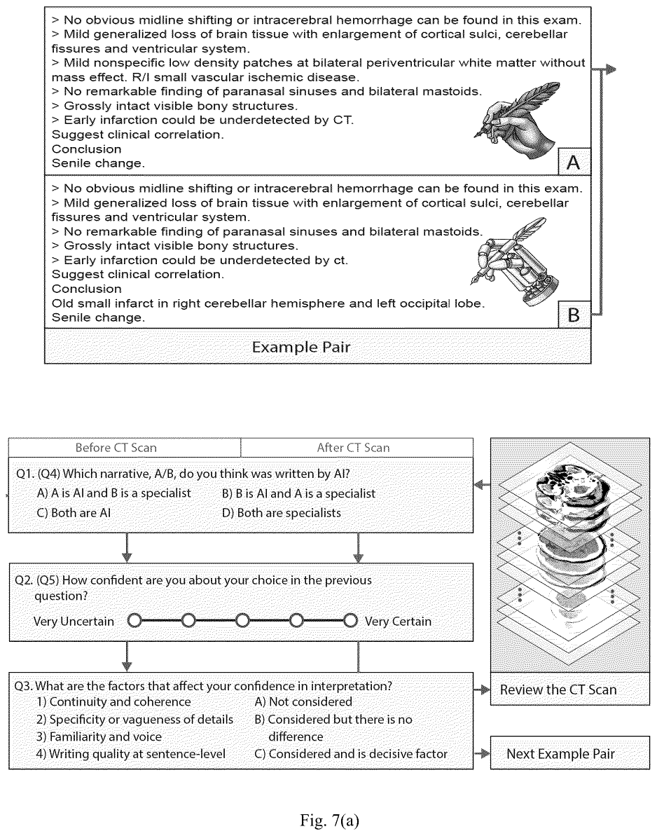

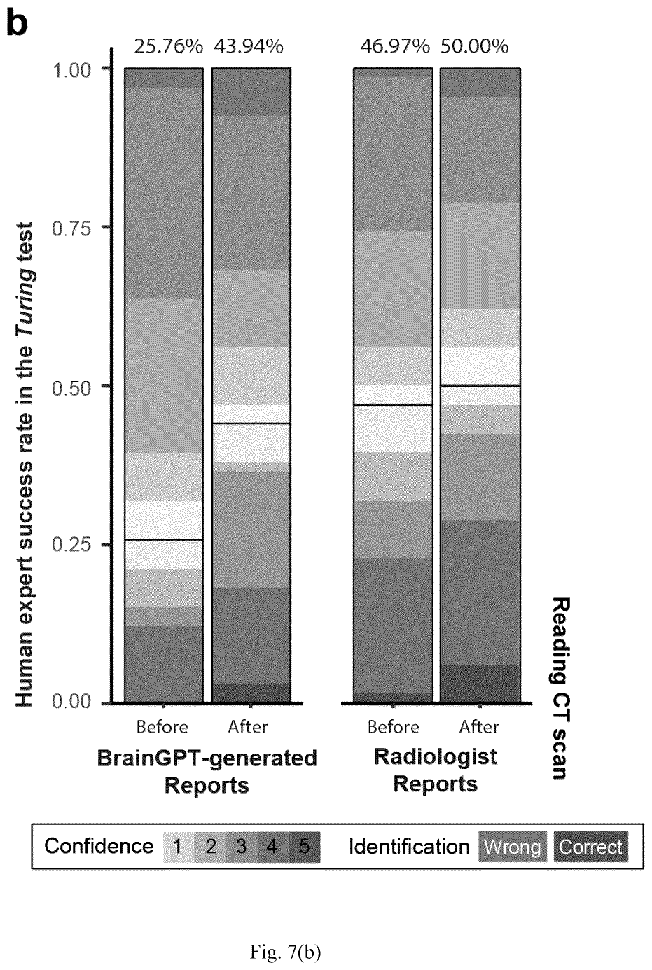

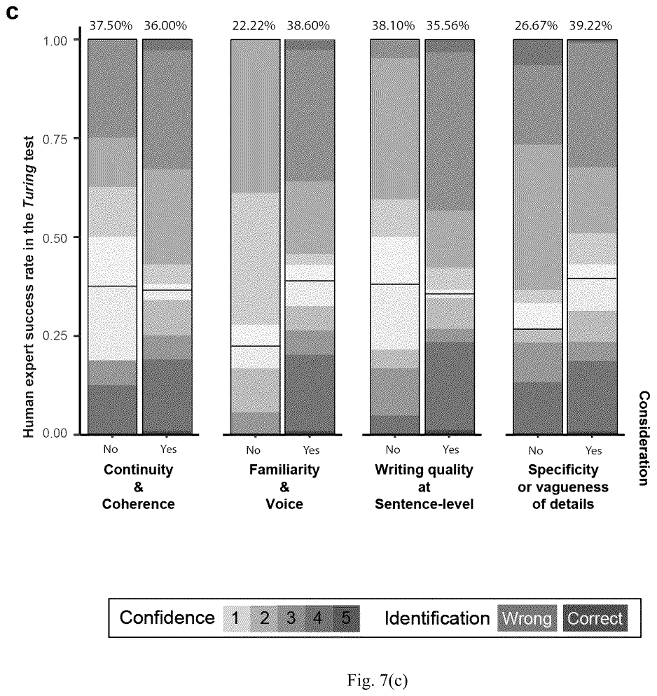

To further evaluate whether healthcare providers could distinguish these BrainGPT-generated reports, we conducted a Turing test. To be specific, we asked 11 physicians (2 radiologists, 2 neurologists, and 7 other licensed medical doctors) (FIG. 7a) to determine whether 6 provided brain CT reports (two lacunar infarcts, cortex atrophy, subdural hemorrhage, and midline shift) were written by machines or humans. Interestingly, out of the 66 evaluations, 74.24% of the BrainGPT-generated captions were mistakenly identified as human-written. (FIG. 7b) When CT input context were provided to the evaluators, 18% of the misidentification was reduced, resulting a 56% incidence of mistake machine-written as human-written reports. Overall, integrating the scan-text context into the Turing test led to more confident evaluations on the 5-point Likert scale (p-value <0.01). This suggests that reviewing actual image contexts helps human evaluators detect subtle differences between machine and human-written reports, likely because of off-target descriptions and inadequate precision in machine-generated captions.

We also investigated what type of qualitative traits can influence the expert impression on the human vs machine originality of the report (FIG. 7c). We found that traits of “Familiarity and voice” (29%), “Specificity or vagueness of details” (37.60%), “Continuity and Coherence” (33.33%), and “Writing quality at sentence level” (33.33%) were significant factors when medical experts attribute a human origin behind a radiology report.³⁴ Notably, when the “Familiarity and voice” factor was taken into account, the average success identification rate increased from 22.22% to 38.6%. Similarly, considering “Specificity or vagueness of details” increased the average success identification rate from 26.67% to 39.22%. By looking on these direct evidence of linguistic-quality embedded expert decision, it is advisable to conduct writing style fine-tuning in terms of RRG.

Together, this study demonstrated a holistic research toward pre-processing 3D volumetric CT image-text pair dataset (negation removal and sentence pairing), fine-tuning a RRG model (BrainGPT) and curating a medical-sensible evaluation metric (FORTE).

### Discussion

In this study, we assembled a holistic framework to address various unmet needs and achieve robust report generation for 3D brain CT. On the basis of our collective experiences, we recommend applying the following sequential protocol (or an equivalent process) to ensure caption and evaluation rigor:
1. CVIT: Incorporating clinical domain knowledge in implementing visual instruction tuning
2. Combining “sentence pairing” and “negation removal”: Adapting traditional metrics to radiology reports
3. Employ FORTE: Gauge the clinical essence of caption results in multiple aspects
4. Leveraging LLM-based agent and human expert evaluation: Measure the medical details of RRG and the keyword-based evaluation accountability
5. Conducting linguistic-embedded Turing tests: Including the corresponding image and linguistic criteria in human expert evaluation

By following these technical approaches, we demonstrated that the fine-tuned open-source MLLMs can be instrumented for 3D medical image interpretation.

Recent work by Hamamci et al. revealed that generative models are competent in 3D chest CT report generation at the state-of-the-art (SOTA) level (BLEU-1=46, BLEU-4=36.9, METEOR=29.5, ROUGE-L=45.9).^{35, 36, 37} However, their customized transformer model required 7 days of training on a single NVIDIA A100 GPU, whereas our BrainGPT model requires only 12 hours of fine-tuning on two NVIDIA A100 GPUs. Additionally, Google AI's Med-Gemini-3D can perform 3D CT report generation, but only 53% of its reports are considered clinically valid in human evaluations.³⁸ The high computational cost of using large-scale Google TPUv4 accelerator pods makes this approach infeasible for general research with limited resources. In contrast, our BrainGPT uses an end-to-end open-source Otter framework (CLIP ViT-L/14 vision encoder³⁹ and LlaMA-7B⁴⁰), which allows for experimental replication and checkpoint sharing. Moreover, the reduced training cost of BrainGPT enables efficient visual instruction tuning, enhancing model performance and tailoring responses to specialized or stylistic requirements.

While we did not modify the Otter model structure, we attribute the SOTA-level performance to the combined effects of RVIT and CVIT. Singhal et al. first explored task-specific RVIT in the medical domain and reported that chatbot performance improved with medical QA in-context example primings.²⁸ Similarly, Med-PaLM M uses image cues (CXR and pathology slides) alongside clinical instructions to guide MLLM on multimodal medical tasks.¹⁷ Echoing these studies, our CVIT models (BrainGPT-template, BrainGPT-keyword) outperformed RVIT models in brain CT captioning. This suggests that fine-grained, specialist-grade instruction design may optimize model outcomes for clinical captioning tasks.

We also highlighted that traditional metrics are unfit for evaluating clinical captioning tasks. Medical image reports assist in differential diagnosis and thus are characterized by complex paraphrasing, high token counts (>100), and numerous negative descriptions, which conflict with common metric evaluation contexts.^{24, 41} Additionally, we observed an “Interpretation Spree” behavior, where BrainGPT provided off-target (but not hallucinated) diagnostic narrations from multiobject brain CT contexts. This behavior is detrimental because (1) off-target effects may exclude the primary disease focus (e.g., stroke or brain tumor), and (2) expanded narration may dilute traditional metrics, leading to invalid evaluations.

To address low traditional metric values from different narration aims, we preprocessed radiology reports with “sentence pairing” and “negation removal.” The application gap is attributed to surface-form similarity and lexicographic analytic restrictions.⁴¹ We then applied FORTE to decompose the BrainGPT reports into keyword categories addressing the effective radiology descriptor frequency. In recent LLM-based evaluation studies^{29, 42, 43, 44}, we found that (1) similar to the sentence pairing process of FORTE, most of them adopt sentence-level comparison when prompting GPT to adapt to the list-by-list nature of radiology reports (2) in coherence with the issue raised in our negation removal session, Doclens²⁹ mentioned the importance of the evaluation method to discriminate “there is a focal lesion” from “there is no focal lesion”; and (3) most importantly, ranging from sentence-level precision and recall to error counting, every study aims to establish a more clinically precise, explainable, and generalizable metric. (Table 2)

To this end, FORTE served as an evaluation framework that consists sentence-pairing, negation removal, and 4-category keyword extraction, which coherently restricts model hallucination, enhance the interpretation convergence and provides instant radiology impressions for healthcare providers. This study performed Pearson correlation analysis across different evaluation metrics, and reported that FORTE method encapsulates a broader medical semantic dimension than the relative unitary traditional metrics. This is further evidenced by its moderate to high correlation with both human expert assessments and DocLens scores.²⁵ Moreover, the FORTE framework is customizable and transferable across various medical tasks with no focus restrain, an interchangeable categorized keyword bank was provided as a keyword JSON files on our GitHub page, some examples were demonstrated for chest X-ray, low-dose computed tomography (LDCT), abdomen CT, and Brain CT tasks. Within the FORTE framework, BrainGPT achieves a commendable performance with an F1 score of 0.589, comparable to the state-of-the-art (SOTA) performance in general medical disease identification, which reports an accuracy of 59.2% in prior benchmark studies.⁴⁵

Human expert evaluation in natural language processing experiments has been conducted under distinct experimental designs and has served diverse study purposes. Therefore, the resulting perspectives were often inconsistent and noncomparable across individual contexts. Therefore, related works have instrumented quantitative (completeness, correctness, conciseness) and qualitative (content, language, structure) measurements to dissect the riveting characters that distinguish synthesized clinical reports from human reports.^{46, 47, 48, 49} By adopting similar designs with objective linguistic criteria, we found that both the reviewer success rate and answer alternation reasons (“Suspicious wording” and “Both not mentioning critical features”) were associated with the writing style (“Familiarity and voice” and “Specificity or vagueness of details”) rather than the sentence-level writing quality and coherence.³⁴ The significance of the medical report writing style was also highlighted in an independent prompting study.⁴⁷ Interestingly, we observed that input case imbalance can influence the caption writing style, potentially related to the over-fitting observed during general model training.

While this invention has been described in connection with what is presently considered to be practical exemplary embodiments, it is to be understood that the invention is not limited to the disclosed embodiments. On the contrary, it is intended to cover various modifications and equivalent arrangements included within the spirit and scope of the appended claims.

### REFERENCES

1. Cao K, et al. Large-scale pancreatic cancer detection via non-contrast CT and deep learning. Nat Med 29, 3033-3043 (2023).
2. Groh M, et al. Deep learning-aided decision support for diagnosis of skin disease across skin tones. Nat Med 30, 573-583 (2024).
3. Chang K J, et al. Rearrange Anatomy Inputs Like LEGO Bricks: Applying InSSS—P and a Mobile-Dense Hybrid Network to Distill Vascular Significance From Retina OCT-Angiography. IEEE Computational Intelligence Magazine 19, 12-25 (2024).
4. Tian F, et al. Prediction of tumor origin in cancers of unknown primary origin with cytology-based deep learning. Nature Medicine, (2024).
5. Dai L, et al. A deep learning system for predicting time to progression of diabetic retinopathy. Nat Med 30, 584-594 (2024).
6. Rajpurkar P, Lungren MP. The Current and Future State of AI Interpretation of Medical Images. N Engl J Med 388, 1981-1990 (2023).
7. Boiselle P M. Computed tomography screening for lung cancer. JAMA 309, 1163-1170 (2013).
8. Wysoki M G, Nassar C J, Koenigsberg R A, Novelline R A, Faro S H, Faerber E N. Head trauma: CT scan interpretation by radiology residents versus staff radiologists. Radiology 208, 125-128 (1998).
9. Boag W, Hsu T-M H, McDermott M, Berner G, Alesentzer E, Szolovits P. Baselines for chest x-ray report generation. In: Machine learning for health workshop). PMLR (2020).
10. Chen Z, Song Y, Chang T-H, Wan X. Generating radiology reports via memory-driven transformer. arXiv preprint arXiv:201016056, (2020).
11. Selivanov A, Rogov O Y, Chesakov D, Shelmanov A, Fedulova I, Dylov D V. Medical image captioning via generative pretrained transformers. Scientific Reports 13, 4171 (2023).
12. Yang S, Wu X, Ge S, Zheng Z, Zhou S K, Xiao L. Radiology report generation with a learned knowledge base and multi-modal alignment. Medical Image Analysis 86, 102798 (2023).
13. Johnson A E W, et al. MIMIC-CXR, a de-identified publicly available database of chest radiographs with free-text reports. Sci Data 6, 317 (2019).
14. Demner-Fushman D, et al. Preparing a collection of radiology examinations for distribution and retrieval. J Am Med Inform Assoc 23, 304-310 (2016).
15. Chen W, Shen L, Li X, Yuan Y. Fine-Grained Image-Text Alignment in Medical Imaging Enables Cyclic Image-Report Generation. Preprint at https://ui.adsabs.harvard.edu/abs/2023arXiv231208078C (2023).
16. Li C, et al. LLaVA-Med: Training a Large Language-and-Vision Assistant for Biomedicine in One Day. Preprint at https://ui.adsabs.harvard.edu/abs/2023arXiv230600890L (2023).
17. Tu T, et al. Towards Generalist Biomedical AI. NEJM AI 1, AIoa2300138 (2024).
18. Haydel M J, Preston C A, Mills T J, Luber S, Blaudeau E, DeBlieux P M. Indications for computed tomography in patients with minor head injury. N Engl J Med 343, 100-105 (2000).
19. König M. Brain perfusion CT in acute stroke: current status. European Journal of Radiology 45, S11-S22 (2003).
20. Banerjee S, Lavie A. METEOR: An automatic metric for MT evaluation with improved correlation with human judgments. In: Proceedings of the acl workshop on intrinsic and extrinsic evaluation measures for machine translation and/or summarization) (2005).
21. Papineni K, Roukos S, Ward T, Zhu W-J. Bleu: a method for automatic evaluation of machine translation. In: Proceedings of the 40^{th} annual meeting of the Association for Computational Linguistics) (2002).
22. Lin C-Y. Rouge: A package for automatic evaluation of summaries. In: Text summarization branches out) (2004).
23. Santos G Od, Colombini E L, Avila S. CIDEr-R: Robust consensus-based image description evaluation. Proceedings of the Seventh Workshop on Noisy User-generated Text (W-NUT 2021), (2021).
24. Liu G, et al. Clinically Accurate Chest X-Ray Report Generation. Preprint at https://ui.adsabs.harvard.edu/abs/2019arXiv190402633L (2019).
25. Irvin J, et al. CheXpert: A Large Chest Radiograph Dataset with Uncertainty Labels and Expert Comparison. Proceedings of the AAAI Conference on Artificial Intelligence 33, 590-597 (2019).
26. Liu H, Li C, Wu Q, Lee Y J. Visual Instruction Tuning. Preprint at https://ui.adsabs.harvard.edu/abs/2023arXiv230408485L (2023).
27. Li B, Zhang Y, Chen L, Wang J, Yang J, Liu Z. Otter: A Multi-Modal Model with In-Context Instruction Tuning. Preprint at https://ui.adsabs.harvard.edu/abs/2023arXiv230503726L (2023).
28. Singhal K, et al. Large language models encode clinical knowledge. Nature 620, 172-180 (2023).
29. Xie Y, et al. DocLens: Multi-aspect Fine-grained Evaluation for Medical Text Generation. Preprint at https://ui.adsabs.harvard.edu/abs/2023arXiv231109581X (2023).
30. Min S, et al. Factscore: Fine-grained atomic evaluation of factual precision in long form text generation. arXiv preprint arXiv:230514251, (2023).
31. Doshi R, Amin K S, Khosla P, Bajaj S S, Chheang S, Forman H P. Quantitative Evaluation of Large Language Models to Streamline Radiology Report Impressions: A Multimodal Retrospective Analysis. Radiology 310, e231593 (2024).
32. Jackson V P, et al. RADPEER scoring white paper. J Am Coll Radiol 6, 21-25 (2009).
33. Wang Z, Luo X, Jiang X, Li D, Qiu L. LLM-RadJudge: Achieving Radiologist-Level Evaluation for X-Ray Report Generation. Preprint at https://ui.adsabs.harvard.edu/abs/2024arXiv240400998W (2024).
34. Casal J E, Kessler M. Can linguists distinguish between ChatGPT/AI and human writing?: A study of research ethics and academic publishing. Research Methods in Applied Linguistics 2, 100068 (2023).
35. Ethem Hamamci I, Er S, Menze B. CT2Rep: Automated Radiology Report Generation for 3D Medical Imaging. Preprint at https://ui.adsabs.harvard.edu/abs/2024arXiv240306801E (2024).
36. Miura Y, Zhang Y, Tsai E B, Langlotz C P, Jurafsky D. Improving factual completeness and consistency of image-to-text radiology report generation. arXiv preprint arXiv:201010042, (2020).
37. Nicolson A, Dowling J, Koopman B. Improving chest X-ray report generation by leveraging warm starting. Artificial Intelligence in Medicine 144, 102633 (2023).
38. Yang L, et al. Advancing Multimodal Medical Capabilities of Gemini. Preprint at https://ui.adsabs.harvard.edu/abs/2024arXiv240503162Y (2024).
39. Radford A, et al. Learning Transferable Visual Models From Natural Language Supervision. Preprint at https://ui.adsabs.harvard.edu/abs/2021arXiv210300020R (2021).
40. Touvron H, et al. LLaMA: Open and Efficient Foundation Language Models. Preprint at https://ui.adsabs.harvard.edu/abs/2023arXiv230213971T (2023).
41. Abbasian M, et al. Foundation metrics for evaluating effectiveness of healthcare conversations powered by generative AI. npj Digital Medicine 7, 82 (2024).
42. Huang A, Banerjee O, Wu K, Pontes Reis E, Rajpurkar P. FineRadScore: A Radiology Report Line-by-Line Evaluation Technique Generating Corrections with Severity Scores. Preprint at https://ui.adsabs.harvard.edu/abs/2024arXiv240520613H (2024).
43. Zambrano Chaves J M, et al. Towards a clinically accessible radiology foundation model: open-access and lightweight, with automated evaluation. Preprint at https://ui.adsabs.harvard.edu/abs/2024arXiv240308002Z (2024).
44. Bannur S, et al. MAIRA-2: Grounded Radiology Report Generation. Preprint at https://ui.adsabs.harvard.edu/abs/2024arXiv240604449B (2024).
45. Liu J, et al. A Spectrum Evaluation Benchmark for Medical Multi-Modal Large Language Models. Preprint at https://ui.adsabs.harvard.edu/abs/2024arXiv240211217L (2024).
46. Van Veen D, et al. Adapted large language models can outperform medical experts in clinical text summarization. Nature Medicine, (2024).
47. Yan B, et al. Style-Aware Radiology Report Generation with RadGraph and Few-Shot Prompting. Preprint at https://ui.adsabs.harvard.edu/abs/2023arXiv231017811Y (2023).
48. Boag W, Kane H, Rawat S, Wei J, Goehler A. A Pilot Study in Surveying Clinical Judgments to Evaluate Radiology Report Generation. In: Proceedings of the 2021 ACM Conference on Fairness, Accountability, and Transparency). Association for Computing Machinery (2021).
49. Youssef A, Pencina M, Thakur A, Zhu T, Clifton D, Shah NH. External validation of AI models in health should be replaced with recurring local validation. Nature Medicine 29, 2686-2687 (2023).
50. Blankemeier L, et al. Merlin: A Vision Language Foundation Model for 3D Computed Tomography. Res Sq, (2024).
51. Chilamkurthy S, et al. Deep learning algorithms for detection of critical findings in head CT scans: a retrospective study. The Lancet 392, 2388-2396 (2018).
52. Lin T-Y, et al. Microsoft COCO: Common Objects in Context. In: Computer Vision—ECCV 2014 (eds Fleet D, Pajdla T, Schiele B, Tuytelaars T). Springer International Publishing (2014).
53. Zhang T, Kishore V, Wu F, Weinberger K Q, Artzi Y. Bertscore: Evaluating text generation with bert. arXiv preprint arXiv:190409675, (2019).
54. Devlin J, Chang M-W, Lee K, Toutanova K. BERT: Pre-training of Deep Bidirectional Transformers for Language Understanding. Preprint at https://ui.adsabs.harvard.edu/abs/2018arXiv181004805D (2018).
55. Feng F, Yang Y, Cer D, Arivazhagan N, Wang W. Language-agnostic BERT Sentence Embedding. Preprint at https://ui.adsabs.harvard.edu/abs/2020arXiv200701852F (2020).
56. Li B, Zhou H, He J, Wang M, Yang Y, Li L. On the Sentence Embeddings from Pre-trained Language Models. Preprint at https://ui.adsabs.harvard.edu/abs/2020arXiv201105864L (2020).
57. Ethayarajh K. How Contextual are Contextualized Word Representations? Comparing the Geometry of BERT, ELMo, and GPT-2 Embeddings. Preprint at https://ui.adsabs.harvard.edu/abs/2019arXiv190900512E (2019).
58. Yu F, et al. Evaluating progress in automatic chest X-ray radiology report generation. Patterns 4, 100802 (2023).
59. Jackson V P, et al. RADPEER™ scoring white paper. Journal of the American College of Radiology 6, 21-25 (2009).

What is claimed is:

1. A system for generation of 3D organ computed tomography (CT) radiology reports, which comprises a 3D-organ CT dataset and a generative pre-trained transformer, which is adapted for a clinically visual instruction-tuned (CVIT) model designed for 3D-organ CT radiology report generation (RRG), and a processor for feature-oriented radiology task evaluation (FORTE).

2. A system for generation of 3D-brain CT radiology reports, comprising a 3D-BrainCT dataset (18,885 text-scan pairs) and a generative pre-trained transformer for the dataset, called as “BrainGPT,” which is adapted for a clinically visual instruction-tuned (CVIT) model designed for 3D-brain CT radiology report generation (RRG), and a processor for feature-oriented radiology task evaluation (FORTE).

3. A process for implementing a multi-modal large language models (MLLM) in 3D organ CT report generation, comprising
collecting a real-world 3D-organ CT dataset;
training BrainGPT models by applying a visual instruction tuning-based approach;
fine-tuning conditions included regular visual instruction tuning (RVIT):
(i) plain instruction (conveying the model's role as a radiology assistant) and
(ii) in-context example instruction (3-shot examples added to the Plain Instruction), or clinical visual instruction tuning (CVIT):
(iii) template instruction (structured clinical-defined QA templates added to the plain instruction) and
(iv) keyword instruction (categorical guidelines focused on keywords added to the plain instruction).
deriving BrainGPT models (-plain, -example, -template, -keyword) that are fine-tuned to perform CT interpretation with different prior levels of clinical sensibility.

4. The process of claim 3, wherein the organ is brain.

5. A comprehensive framework, comprising dataset curation, anatomy-aware model fine-tuning, and the development of robust evaluation metrics for the RRG.
